(19) United States
(12) Patent Application Publication
(10) Pub. No.: US 2017/0157012 A1
(43) Pub. Date: Jun. 8, 2017

(54) A Use of Isosorbide Monooleate

(71) Applicant: BASF SE, Ludwigshafen (DE)

(72) Inventors: Claudia Stoer, Solingen (DE); Claus Nieendick, Krefeld (DE); Markus Weissenegger, Düsseldorf (DE); Daniela Printz, Dormagen (DE); Mirella Winzek, Titz (DE); Jennifer Schoss, Erkrath (DE); Markus Dierker, Düsseldorf (DE); Norbert Boyxen, Kempen (DE); Ute Griesbach, Düsseldorf (DE); Werner Seipel, Hilden (DE); Werner Mauer, Sendenhorst-Albersloh (DE)

(73) Assignee: BASF SE, Ludwigshafen (DE)

(21) Appl. No.: 15/325,463

(22) PCT Filed: Jul. 6, 2015
(86) PCT No.: PCT/EP2015/065326

§ 371 Date: Jan. 11, 2017

(30) Foreign Application Priority Data
Jul. 16, 2014 (EP) 14177258.2

(51) Int. Cl. A61K 8/49; A61Q 19/10; A61Q 5/02

(52) U.S. Cl. CPC A61K 8/4973; A61Q 5/02; A61Q 19/10

(57) ABSTRACT

The present invention relates to the use of a cosmetic composition comprising isosorbide monooleate for depositing isosorbide monooleate on human skin or hair. Furthermore it relates to a process for depositing isosorbide monooleate on human skin or hair, wherein this process comprises contacting human skin or hair with a cosmetic composition comprising isosorbide monooleate and rinsing off the cosmetic composition comprising isosorbide monooleate with water, so that, after rinsing off the cosmetic composition, isosorbide monooleate is left deposited on the human skin or hair.

The present invention relates to the use of a cosmetic composition comprising isosorbide monooleate for depositing isosorbide monooleate on human skin or hair. Furthermore it relates to a process for depositing isosorbide monooleate on human skin or hair, wherein this process comprises contacting human skin or hair with a cosmetic composition comprising isosorbide monooleate and rinsing off the cosmetic composition comprising isosorbide monooleate with water, so that, after rinsing off the cosmetic composition, isosorbide monooleate is left deposited on the human skin or hair.

Cosmetic compositions, detergents and cleansers comprising isosorbide monooleate, i. e. the monoester of isosorbide and oleic acid, are known. Isosorbide monooleate has many advantageous properties which are the reason why isosorbide monooleate is used in cosmetic compositions, in detergents and in cleansers.

WO 2010/115565 (EP 2 239 315) discloses detergents and cleansers comprising isosorbide monoesters. In the example section a composition comprising 1% by weight isosorbide monoester and 3% by weight coco glucoside is disclosed.

WO 2013/041388 discloses cosmetic compositions comprising isosorbide monooleate, specifically disclosed is a composition comprising 1% by weight isosorbide monooleate and 1.5% by weight coco glucoside (FIG. 4). This composition has good foaming performance.

European patent application no. 14154978 filed on Feb. 13, 2014 (BASF internal file no. PF 75368) discloses a microemulsion comprising isosorbide monooleate and caprylyl/capryl glucoside and lauryl glucoside. It is disclosed that isosorbide monooleate which is used commercially is generally a mixture of monoester, diester, isosorbide and fatty acids, mainly oleic acid.

The treatment of human skin or hair with a cosmetic composition, e. g. a shampoo or a shower gel or a body wash composition, can lead to a depletion of the skin or hair of protecting lipids. This can lead to dry skin, to brittle hair, and to other unwanted effects.

The problem underlying the present invention is to avoid or reduce these negative effects.

This problem is solved by the use of a cosmetic composition comprising isosorbide monooleate for depositing isosorbide monooleate on human skin or hair. This use is a subject of the present invention.

One embodiment of the present invention is the use according to the present invention, wherein the cosmetic composition comprises isosorbide monooleate in an amount of 0.2 to 3% by weight, preferably 0.4 to 2.5% by weight, more preferably 0.5 to 2% by weight.

One embodiment of the present invention is the use according to the present invention (i. e. according to the use which is the subject of the present invention or according to any of the embodiments described in the previous paragraphs), wherein the cosmetic composition comprises a surfactant which is different from isosorbide monooleate selected from the group consisting of an anionic surfactant, a cationic surfactant, a nonionic surfactant an amphoteric or zwitterionic surfactant and mixtures thereof, preferably in an amount of 2 to 40% by weight, more preferably 5 to 30% by weight, more preferably 10 to 20% by weight.

One embodiment of the present invention is the use according to the embodiment described in the previous paragraph, wherein the surfactant which is different from isosorbide monooleate is an anionic surfactant and wherein the cosmetic composition further comprises a cationic polymer suitable for use in a cosmetic composition, preferably in an amount of 0.1 to 1% by weight, more preferably 0.1 to 0.8% by weight, more preferably 0.15 to 0.5% by weight, and wherein the cationic polymer is preferably selected from the group consisting of a polyquaternium (according to INCI nomenclature) and a cationic guar derivative, more preferably selected from the group consisting of polyquaternium 7, polyquaternium 10 and a cationic guar derivative.

One embodiment of the present invention is the use according to the present invention (i. e. according to the use which is the subject of the present invention or according to any of the embodiments described in the previous paragraphs), wherein the cosmetic composition further comprises one or more cosmetically acceptable ingredients.

One embodiment of the present invention is the use according to the present invention (i. e. according to the use which is the subject of the present invention or according to any of the embodiments described in the previous paragraphs), wherein the cosmetic composition is selected from the group consisting of a shampoo, a shower gel, a body-wash composition and a skin or hair conditioner.

One embodiment of the present invention is the use according to the present invention (i. e. according to the use which is the subject of the present invention or according to any of the embodiments described in the previous paragraphs), wherein this use results in a lipid layer enhancing effect on the skin or hair or in a moisturizing effect on the skin or hair or in a humectant effect on the skin or hair or in a protecting effect on the skin or hair or in a caring effect on the skin or hair.

One embodiment of the present invention is the use according to the present invention (i. e. according to the use which is the subject of the present invention or according to any of the embodiments described in the previous paragraphs), wherein the isosorbide monooleate is a mixture comprising pure isosorbide monooleate in an amount of 65-95% by weight, preferably 65-85% by weight, more preferably 65-75% by weight.

One embodiment of the present invention is the use according to the embodiments described in the previous paragraph, wherein the fatty acid moieties in the pure isosorbide monooleate comprise more than 65% by weight, preferably more than 70% by weight, more preferably more than 75% by weight, oleic acid moieties.

One embodiment of the present invention is the use according to the present invention (i. e. according to the use which is the subject of the present invention or according to any of the embodiments described in the previous paragraphs), wherein the use is realized in a process comprising contacting human skin or hair with a cosmetic composition comprising isosorbide monooleate and rinsing off the cosmetic composition comprising isosorbide monooleate with water, so that, after rinsing off the cosmetic composition, isosorbide monooleate is left deposited on the human skin or hair, preferably in an amount of 0.01 to 10 μg/cm², more preferably 0.1 to 5 μg/cm², more preferably 0.5 to 1 μg/cm².

Another subject of the present invention is a process for depositing isosorbide monooleate on human skin or hair, wherein this process comprises contacting human skin or hair with a cosmetic composition comprising isosorbide monooleate and rinsing off the cosmetic composition comprising isosorbide monooleate with water, so that, after rinsing off the cosmetic composition, isosorbide monooleate is left deposited on the human skin or hair.

One embodiment of the present invention is the process according to the present invention, wherein the isosorbide monooleate is left deposited on the human skin or hair in an amount of 0.01 to 10 μg/cm², preferably 0.1 to 5 μg/cm², more preferably 0.5 to 1 μg/cm².

One embodiment of the present invention is the process according to the present invention (i. e. according to the process which is the subject of the present invention or according to any of the embodiments described in the previous paragraphs), wherein the cosmetic composition is a cosmetic composition as defined in the embodiments of the use according to the present invention.

One embodiment of the present invention is the process according to the present invention (i. e. according to the process which is the subject of the present invention or according to any of the embodiments described in the previous paragraphs), wherein this process results in a lipid layer enhancing effect on the skin or hair or in a moisturizing effect on the skin or hair or in a humectant effect on the skin or hair or in a protecting effect on the skin or hair or in a caring effect on the skin or hair.

One embodiment of the present invention is the process according to the present invention (i. e. according to the process which is the subject of the present invention or according to any of the embodiments described in the previous paragraphs), wherein the isosorbide monooleate is an isosorbide monooleate as defined in the embodiments of the use according to the present invention.

Isosorbide monooleate can be made as disclosed in WO 2010/115565 or by other known esterification methods.

Cosmetic compositions comprising isosorbide monooleate can be made as disclosed in WO 2013/041388 or by other known methods for making cosmetic compositions.

Isosorbide monooleate according to the present invention generally is a mixture comprising (pure) isosorbide monooleate as major component and additionally isosorbide dioleate, oleic acid and isosorbide. In this mixture the oleic acid, which is generally obtained from plants, also is a mixture comprising oleic acid as major component and other fatty acids in minor amounts. The term pure isosorbide monooleate means the monoester of isosorbide with a fatty acid mixture comprising oleic acid as major component. Here, major component means more than 65% by weight, preferably more than 70% by weight, more preferably more than 75% by weight, oleic acid. Therefore, in one embodiment of the present invention the isosorbide monooleate is a mixture comprising pure isosorbide monooleate in an amount of 65-95% by weight, preferably 65-85% by weight, more preferably 65-75% by weight. The amounts in % by weight can be determined by gas chromatography (GC) with appropriate calibration. In this pure isosorbide monooleate the fatty acid moieties comprise more than 65% by weight, preferably more than 70% by weight, more preferably more than 75% by weight, oleic acid moieties. The amounts in % by weight can be determined by gas chromatography (GC) with appropriate calibration.

Cosmetic compositions are to be understood here as meaning all compositions which are exclusively or primarily intended to be used externally on the human body or in its oral cavity for cleaning, care, protection, maintaining a good condition, perfuming, changing the appearance or for the purposes of influencing body odor.

The cosmetic compositions according to the invention can be in particular formulations for bodycare, e.g. a body milk, creams, lotions, sprayable emulsions, products for eliminating body odor etc. They can be surfactant-containing formulations such as e.g. foam and shower baths, hair shampoos and care rinses. Depending on the intended application, the cosmetic formulations can comprise a series of further auxiliaries and additives, for example surfactants, oil bodies, emulsifiers, pearlescent waxes, consistency regulators, thickeners, superfatting agents, stabilizers, polymers, fats, waxes, lecithins, phospholipids, biogenic active ingredients, UV light protection factors, antioxidants, deodorants, antiperspirants, antidandruff agents, film formers, swelling agents, insect repellents, self-tanning agents, tyrosine inhibitors (depigmentation agents), hydrotropes, solubilizers, preservatives, perfume oils, dyes etc., which are listed below by way of example.

Surfactants Surface-active substances which may be present are anionic, nonionic, cationic and/or amphoteric or zwitterionic surfactants. In surfactant-containing cosmetic preparations, for example shower gels, foam baths, shampoos etc., preferably at least one anionic surfactant is present. The fraction of surfactants here is usually about 1 to 30, preferably 5 to 25 and in particular 10 to 20% by weight.

Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglycoside sulfates, protein fatty acid condensates (in particular wheat-based vegetable products) and alkyl (ether) phosphates. If the anionic surfactants contain polyglycol ether chains, these can have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants contain polyglycol ether chains, these can have a conventional homolog distribution, but preferably have a narrowed homolog distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetaines, alkylamidobetaines, aminopropionates, aminoglycinates, imidazoliniumbetaines and sulfobetaines. Said surfactants are exclusively known compounds. As regards structure and preparation of these substances, reference may be made to relevant review works in this field. Typical examples of particularly suitable mild, i.e. particularly skin-compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter preferably based on wheat proteins.

Oil bodies: Bodycare compositions, such as creams, lotions and milks, usually comprise oil bodies and emollients. Suitable oil bodies and emollients are disclosed in WO 2013/041388.

Fats and waxes: Fats and waxes are added to bodycare products as care substances and also in order to increase the consistency of the cosmetics. Suitable fats and waxes are disclosed in WO 2013/041388.

Suitable thickeners are disclosed in WO 2013/041388.

UV light protection factors are to be understood as meaning, for example, organic substances (light protection filters) that are present in liquid or crystalline form at room temperature and which are able to absorb ultraviolet rays and release the absorbed energy again in the form of longer-wave radiation, e.g. heat. Suitable UV light protection factors are disclosed in WO 2013/041388.

Biogenic active ingredients are to be understood as meaning, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g. prune extract, bambara nut extract and vitamin complexes.

Deodorizing active ingredients counteract, mask or eliminate body odors. Suitable deodorizing active ingredients are disclosed in WO 2013/041388.

Suitable insect repellents, self-tanning agents, tyrosine inhibitors, preservatives, perfumes, pearlescent waxes, superfatting agents, stabilizers, hydrotropes, which can be present in the composition according to the present invention are disclosed in WO 2013/041388.

### EXAMPLES

For measuring the deposition of isosorbide monooleate on a substrate a formulation containing isosorbide monooleate was applied to this substrate. Then, the treated area was rinsed with a defined amount of water and extracted with an organic solvent in order to determine the amount of isosorbide monooleate that remained on the surface after the rinse cycle. The extract was analyzed by gas chromatography (GC) and the amount of isosorbide monooleate deposited on the surface was calculated. The measurements were carried out as described in the experimental section of WO 2013/007473.

As substrate the human skin (forearm) of probands (in-vivo deposition) as well as a skin substitute (collagen layer) (in-vitro deposition) was used. The rinse-cycle (dilution with water) imitated the process of having a shower.

The following formulations were used for the deposition measurements (in-vitro as well as in-vivo). The isosorbide monooleate had a content of about 77% by weight monoester in which the acid moiety had a content of more than 75% by weight oleic acid moieties.

### Composition of Formulations Tested

| | Amount | Amount | Amount | Amount |
|---|---|---|---|---|
| Components | (% by | (% by | (% by | (% by |
| (INCI) | weight) | weight) | weight) | weight) |
| Sodium Laureth Sulfate | 11.2 | 11.2 | 11.2 | 11.2 |
| Sodium Chloride | 1.0-1.8 | 1.0-1.8 | 1.0-1.8 | 1.0-1.8 |
| Cocoamidopropyl | 1.5 | 1.5 | 1.5 | 1.5 |
| Betaine | | | | |
| Natriumbenzoat | 0.5 | 0.5 | 0.5 | 0.5 |
| Polyquaternium-7 | 0.2 | 0.2 | 0.2 | 0.2 |
| Coco Glucoside | 1.5 | 1.5 | 1.5 | 1.5 |
| Isosorbide Monooleate | 0.5 | 1 | 1.5 | 2 |
| Citric acid | 0.2 | 0.2 | 0.2 | 0.2 |
| water | Up to 100 | Up to 100 | Up to 100 | Up to 100 |

### In-Vitro Deposition: Results

A concentration dependency was observed: The higher the amount of isosorbide monooleate in the formulation the higher the deposition rate of this substance on the skin/skin substitute.

| Concentration of | In-vitro deposition of |
|---|---|
| isosorbide monooleate | Isosorbide monooleate |
| in the formulation | on the skin substitute |
| (% by weight) | per area (μg/cm²) |
| 0.5 | 3.7 |
| 1 | 7.3 |
| 1.5 | 8.6 |
| 2 | 15.8 |

### In-Vivo Deposition: Results

The formulation with 1% by weight isosorbide monooleate was tested. It could be shown that isosorbide monooleate remained in small amounts on the skin after rinse-off.

| Concentration of | In-vivo Deposition of |
|---|---|
| isosorbide monooleate | isosorbide monooleate |
| in the formulation | on the skin per area |
| (% by weight) | (μg/cm²) |
| 1 | 0.7 |

1. (canceled)

2. The process according to claim 11, wherein the cosmetic composition comprises isosorbide monooleate in an amount of 0.2 to 3% by weight.

3. The process according to claim 1, wherein the cosmetic composition comprises a surfactant different from isosorbide monooleate selected from the group consisting of an anionic surfactant, a cationic surfactant, a nonionic surfactant, an amphoteric or zwitterionic surfactant, and mixtures thereof, in an amount of 2 to 40% by weight.

4. The process according to claim 3, wherein the surfactant is different from isosorbide monooleate is an anionic surfactant and wherein the cosmetic composition further comprises a cationic polymer suitable for use in a cosmetic composition, in an amount of 0.1 to 1% by weight.

5. The process according to claim 2, wherein the cosmetic composition further comprises one or more cosmetically acceptable ingredients.

6. The process according to claim 11, wherein the cosmetic composition is selected from the group consisting of a shampoo, a shower gel, a body-wash composition, and a skin or hair conditioner.

7. (canceled)

8. The process according to claim 11, wherein the isosorbide monooleate is a mixture comprising pure isosorbide monooleate in an amount of 65-95% by weight.

9. The process according to claim 8, wherein the fatty acid moieties in the pure isosorbide monooleate comprise more than 65% by weight oleic acid moieties.

10. (canceled)

11. A process for depositing isosorbide monooleate on human skin or hair, wherein the process comprises
contacting human skin or hair with a cosmetic composition comprising isosorbide monooleate, and
rinsing off the cosmetic composition comprising isosorbide monooleate with water, so that, after rinsing off the cosmetic composition, isosorbide monooleate is left deposited on the human skin or hair.

12. The process according to claim 11, wherein the isosorbide monooleate is left deposited on the human skin or hair in an amount of 0.01 to 10 μg/cm².

13. (canceled)

14. The process according to claim 11, wherein the process results in a lipid layer enhancing effect on the skin or hair or in a moisturizing effect on the skin or hair or in a humectant effect on the skin or hair or in a protecting effect on the skin or hair or in a caring effect on the skin or hair.

15. (canceled)

16. The process according to claim 4, wherein the cationic polymer is selected from the group consisting of a polyquaternium and a cationic guar derivative.

17. The process according to claim 4, wherein the cationic polymer is selected from the group consisting of polyquaternium 7, polyquaternium 10, and a cationic guar derivative.

18. The process according to claim 8, wherein the isosorbide monooleate is a mixture comprising pure isosorbide monooleate in an amount of 65-85% by weight.

19. The process according to claim 9, wherein the fatty acid moieties in the pure isosorbide monooleate comprise more than 70% by weight oleic acid moieties.

20. The process according to claim 12, wherein the isosorbide monooleate is left deposited on the human skin or hair in an amount of 0.1 to 5 μg/cm².

21. The process according to claim 20 wherein, the isosorbide monooleate is left deposited on the human skin or hair in an amount of 0.5 to 1 μg/cm².
